(19) United States
(12) Patent Application Publication
(10) Pub. No.: US 2004/0137462 A1
(43) Pub. Date: Jul. 15, 2004

(54) Control sets of target nucleic acids and their use in array based hybridization assays

(76) Inventors: Alex Chenchik, Palo Alto, CA (US); Alexander Munishkin, Santa Cruz, CA (US)

Correspondence Address: BOZICEVIC, FIELD & FRANCIS (BD BIOSCIENCES), 200 MIDDLEFIELD ROAD, SUITE 200, MENLO PARK, CA, 94025, US

(21) Appl. No.: 10/602,550

(22) Filed: Jun. 23, 2003

Related U.S. Application Data
(63) Continuation of application No. 09/750,452, filed on Dec. 27, 2000, now abandoned.
Application No. 09/750,452 is a continuation-in-part of application No. 09/298,361, filed on Apr. 23, 1999, now abandoned.

(51) Int. Cl. C12Q 1/68; C12M 1/34

(52) U.S. Cl. 435/6; 435/287.2

(57) ABSTRACT

Methods for performing array-based hybridization assays, as well as sets of control target nucleic acids for use in the subject methods and kits comprising the same, are provided. In the subject methods, test and control sets of target nucleic acids are contacted with an array(s), where the test and control sets of target nucleic acids may or may not be labeled differentially. The subject control sets of nucleic acids contain a plurality of distinct target nucleic acids, where at least a portion of the probes on the array are represented in the control set of target nucleic acids.

### CROSS-REFERENCE TO RELATED APPLICATIONS

This application is a continuation of application Ser. No. 09/750,452, filed on Dec. 27, 2000, which is a continuation-in-part of application Ser. No. 09/298,361 filed on Apr. 23, 1999, now abandoned; the disclosure of which is herein incorporated by reference.

### INTRODUCTION

1. Technical Field

The field of this invention is nucleic acid arrays.

2. Background of the Invention

“Biochips” or arrays of binding agents, such as oligonucleotides and peptides, have become an increasingly important tool in the biotechnology industry and related fields. These binding agent arrays, in which a plurality of binding agents are deposited onto a support surface, often a solid support surface, in the form of an array or pattern, find use in a variety of applications, including drug screening, nucleic acid sequencing, mutation analysis, and the like. One important use of biochips is in the analysis of differential gene expression, where the expression of genes in different cells, normally a cell of interest and a control, is compared and any discrepancies in expression are identified. In such assays, the presence of discrepancies indicates a difference in the classes of genes expressed in the cells being compared.

In methods of differential gene expression, arrays find use by serving as a substrate to which is bound polynucleotide “probe” fragments. One then obtains “targets” from analogous cells, tissues or organs of a healthy and diseased organism. The targets are then hybridized to the immobilized set of polynucleotide “probe” fragments. Differences between the resultant hybridization patterns are then detected and related to differences in gene expression in the two sources. While the terms “probe” and “target” have been used in different manners in the literature, as used herein and in accordance-with the Nature Genetics Supplement, Vol. 21, published January 1999, the term “probe” refers to the “tethered” nucleic acid while the term “target” refers to the nucleic acid in solution.

Because of the varied and important information that arrays can provide, as well as the many potential applications of such devices, the use of arrays in research, diagnostic and related applications has grown considerably and is expected to continue to do so. A variety of different array technologies have been developed in order to meet the growing need of the biotechnology industry.

However, there are disadvantages with current protocols. For example, the efficiency of hybridization of target nucleic acids to the array can be limited by experimental or physical limitations, e.g., different target nucleic acids can have different hybridization efficiencies to the probe nucleic acids of the array. Differences in hybridization efficiency result in differences in the intensity of hybridization to different probe nucleic acids of the array, even though the targets are present in equivalent concentrations. In addition, differences in the quality of the probes on the array (e.g., probe purity, size differences, presence of impurities, etc.) can give different signals. Alternatively, where two arrays are employed in a particular application, e.g., in gene expression analysis, variation in the quality of array (reproducibility of array production), and in assay conditions between the different arrays can preclude direct comparison of data obtained on the arrays, since conditions such as hybridization time, probe labeling, detection procedures, etc., may differ, and variations between the arrays may be present.

Furthermore, it is difficult to compare data generated using different types of oligonucleotide or polynucleotide-based arrays. Concentration of target nucleic acids in a sample cannot be compared between arrays produced by different methods and/or manufacturers based on intensity of signals because the set of probe sequences often differs between arrays.

As a result, current array technology is used mainly for discovery of differentially-expressed genes rather than for any specific quantitative assay. Two formats are generally employed: (a) comparison of two hybridization patterns to each other and (b) simultaneous hybridization to the same array of two different targets derived from two different biological sources and labeled by different labels. In the latter approach, which is more commonly employed, fold differences in gene expression between the two samples are often measured.

Quantitative array based gene expression analysis protocols have been reported in the literature. For example, U.S. Pat. No. 6,040,138 reports quantitative gene expression analysis using an array and a fluorescent label. Quantitation is provided by the inclusion of control probe sequences, e.g., probes to one or more housekeeping genes, such as the transferrin receptor gene and the GADPH gene. See also U.S. Pat. No. 5,807,522; U.S. Pat. No. 5,830,645 and Schena et al., Science (Oct. 20, 1995) 270:467; which also discuss or imply the use of housekeeping control genes or other specific control probe sequences for use array based in quantitative gene expression analysis protocols.

A problem with the above control probe based protocols is that they do not provide an “internal control” for each probe spot on the array and use only one control RNA in order to “calibrate” other probes on the array. This approach is therefore based on the incorrect assumption that all of the probes on the array have the same hybridization characterization. As such, miscalculations in quantities of gene expression derived from such protocols can be made.

As such, there is a continued need for the development of alternative calibration technologies. Of particular interest would be the development of an array-based methodology that incorporates an internal calibration standard, where such a method would eliminate variations resulting from the quality of the array and the nature of the probes on the array, the type of the array, the quality of the assay conditions, and the like. In addition, there is a need for an array-based protocol that provides quantitative data about target concentration, and a corresponding method of quantification to allow more accurate comparison of data between arrays.

Relevant Literature

U.S. Patents of interest are: U.S. Pat. Nos. 6,040,138; 5,807,522 and 5,830,645. A review of the state of the art in array based hybridization assays is provided in Nature Genetics Supplement (January 1999) Vol. 21. See also Schena et al., Science (Oct. 20, 1995) 270:467.

### SUMMARY OF THE INVENTION

Methods for performing array-based hybridization assays, as well as sets of nucleic acids for use as internal calibration standards and kits comprising the same, are provided. A feature of the subject methods is that a control set of target nucleic acids is used in addition to the test set of target nucleic acids. The control set of target nucleic acids employed in the subject methods contains a plurality of distinct nucleic acids of different sequence capable of selectively binding to at least a subset of, if not all of, the probe compositions present on the array with which it is used, e.g., at least a subset or portion of the probe nucleic acids present on the array are represented in the control set. In the subject methods, the test and control sets of target nucleic acids may be hybridized to the same or different arrays, where the control and test set of target nucleic acids may be labeled the same or differently, depending on the particular protocol, e.g., whether the two sets are hybridized sequentially, whether a single array or two arrays are employed, etc. Also provided are sets of control nucleic acids for use in the subject methods, as well kits for use in performing the subject methods. The subject methods and compositions find particular use in quantitative differential gene expression analysis, both on a single array and for comparison of data between arrays.

### DEFINITIONS

The term “nucleic acid” as used herein means a polymer composed of nucleotides, e.g., deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides in length.

The term “polynucleotide” as used herein refers to single or double-stranded polymer composed of nucleotide monomers of greater than about 120 nucleotides in length up to about 1000 nucleotides in length.

The term “array” refers to a plurality of nucleic acids stably associated with the surface of a solid support, where at least a portion of the stably associated nucleic acids are probe nucleic acids.

The term “probe” means a nucleic acid that is stably associated with, e.g., tethered to or otherwise immobilized on, a solid support surface, such as a planar surface of an array substrate.

The term “probe nucleic acid” refers to nucleic acids stably associated with the surface of a solid support which correspond to a target gene of interest in a sample. Probe nucleic acids are not random nucleic acids or nucleic acids that correspond to genes that are not of interest in a sample, e.g. housekeeping genes, genes that are widely expressed among tissues, etc.

The term “target” means a nucleic acid free in solution.

The term “target nucleic acid” refers to a nucleic acid isolated from a physiological sample which comprises a gene(s) of interest.

The term “control target nucleic acid” refers to a nucleic acid that is complementary to a probe nucleic acid on an array, where the control target nucleic acid is part of a set of target nucleic acids in which the amount of each nucleic acid in the set is known and in which each probe nucleic acid corresponding to a gene of interest present on an array is represented. Control target nucleic acid is (in a preferred embodiment) synthetic RNA, or nucleic acid derived therefrom, like synthetic oligonucleotides or cDNA, made artificially in vitro and not isolated from a biological source. As such, in a preferred embodiment control target nucleic acid is distinguished from test target and test control, which are derived from a biological source. Control target nucleic acids are not the same as test control nucleic acids, which are derived from the same physiological sample as the test target nucleic acids and are therefore unique and specific for that sample. A further contrast been test control nucleic acids and the subject control target nucleic acids is that test control nucleic acids do not correspond, e.g., they are not complementary to, probe nucleic acids on an array, but instead correspond to non-probe nucleic acids on the array, e.g. housekeeping genes, etc.

### DESCRIPTION OF THE SPECIFIC EMBODIMENTS

Methods and compositions for performing quantitative array-based hybridization assays are provided. In the subject methods, both a test set of target nucleic acids and a control set of target nucleic acids are employed, where the control set of target nucleic acids is characterized by including a plurality of distinct nucleic acids of different sequence that bind selectively to probe nucleic acids in at least a subset of the probe nucleic acids present on the array, i.e., at least a subset of, if not all of, the probe nucleic acids present on the array employed in the method is represented in the control set. Depending on the protocol employed, e.g., whether a single array is employed, whether the control and test sets are hybridized sequentially or simultaneously to the same array, etc., the control and test sets of target nucleic acids may be labeled with the same label or differentially labeled, i.e., labeled such that they are simultaneously distinguishable from each other. Also provided are sets of control target nucleic acids for use in the subject methods, as well kits comprising the sets of control target nucleic acids for use in performing the subject methods. The subject invention finds use in a variety of different applications, including gene expression analysis. In further describing the subject invention, the methods will be described first, followed by a description of the subject kits and a discussion of representative applications in which the subject invention finds use.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural references unless the context clearly dictates otherwise. Unless defined otherwise, all technical and scientific terms used herein have the same meaning as commonly understood to one of ordinary skill in the art to which this invention belongs.

METHODS

As summarized above, the subject invention is directed to array-based hybridization assays in which a test set of labeled target nucleic acids is hybridized to an array of probe nucleic acids. A feature of the subject invention is the use of a control set of target nucleic acids, where at least a subset of the probe nucleic acids, and in certain preferred embodiments all of the probe nucleic acids, present on the array are represented in the control set, i.e., the control set includes a nucleic acid capable of hybridizing to each different probe nucleic acid of at least a subset of all of the different probe nucleic acids of the array with which it is employed. In general, the subject methods include the following steps: (1) procurement of the test and control target nucleic acids; (2) hybridization to an array(s); (3) washing; and (4) detection. Each of these steps of the subject methods is described in greater detail below.

Procurement of Control and Test Target Nucleic Acids

The first step in the subject methods is the procurement of the labeled control and test sets of target nucleic acids. Both the control and test sets of nucleic acids are pools, mixtures or collections of a plurality of distinct nucleic acids that differ by sequence.

With respect to the control set of target nucleic acids, the number of distinct nucleic acids which differ from each other in terms of sequence (i.e., any two distinct nucleic acids have a different nucleotide sequence) in the control set of target nucleic acids is at least about 20, usually at least about 50, more usually at least about 100 and often at least about 200, where the number of distinct nucleic acids in a given set may be as high as 20,000 or higher, but will typically not exceed about 10,000 and usually will not exceed about 5,000. As such, with respect to the control set of target nucleic acids, the term “plurality” means at least about 20.

With respect to the test set of target nucleic acids, the number of distinct nucleic acids in the test set is generally at least about 1000 and generally less than about 50,000, where the number generally ranges from about 5,000 to 20,000. The control and test sets of target nucleic acids are now described separately in greater detail.

Control Sets of Target Nucleic Acids and Sets Thereof

The control sets of target nucleic acids are collections or pools of control target nucleic acids (generally at least 50 distinct nucleic acids of different sequence), as mentioned above. A control target nucleic acid may be any nucleic acid capable of hybridizing selectively to its respective probe nucleic acid on an array, typically under stringent conditions, where representative stringent conditions are described infra. As such, the nucleotides that make up the control target nucleic acid molecule may be made up of nucleotides that are naturally occurring or nucleotides that are synthetically produced analogues that are capable of forming base-pair relationships with naturally occurring base pairs.

As such, the control target nucleic acids may include polymers of ribonucleotides and deoxyribonucleotides, with the ribonucleotide and/or deoxyribonucleotides being connected together via 5′ to 3′ linkages. Control nucleic acids of the invention may be ribonucleic acids, for example sense or antisense ribonucleic acids, full-length or partial fragments of cRNA, full-length or partial fragments of mRNA, and/or oligoribonucleotides. Alternatively, control target nucleic acids of the invention may be deoxyribonucleic acids, preferably single-stranded full-length or fragments of sequences encoding the corresponding mRNAs, e.g., first strand cDNA. As such, the nucleic acids of the control set may be RNAs or derivatives thereof, e.g., cDNAs reverse transcribed from RNAs or synthetic DNA oligonucleotides. In one preferred embodiment, the control set is a mixture of synthetic oligonucleotides complementary to probe sequences on the array. The form of the control and target nucleic acids should be chosen so that they are complimentary to and form appropriate Watson-Crick hydrogen bonds with probes present in an array with which the particular control set is to be employed. For example if probe sequences correspond in sequence to mRNA, then target sequences should be complementary, e.g., antisense or complementary RNA (cRNA).

As mentioned above, the control target nucleic acids may be polymers of synthetic nucleotide analogs. Such control target nucleic acids may be preferred in certain embodiments because of their superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O=phosphorothioate, 3′-CH₂-5′-O-phosphonate and 3′-NH-5′-O-phosphoroamidate. Peptide nucleic acids replace the entire ribose phosphodiester backbone with a peptide linkage. Locked nucleic acids give additional conformational stability of sugar moiety due to additional bonds between 2′-carboxil and 5′-carboxil or 4′-carboxil groups of deoxyribose. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases that find use in the method of the invention are those capable of appropriate base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively. Examples of non-naturally occurring bases that are capable of forming base-pairing relationships include, but are not limited to, aza and deaza pyrimidine analogues, aza and deaza purine analogues, and other heterocyclicbase analogues, wherein one or more of the carbon and nitrogen atoms of the purine and pyrimidine rings have been substituted by heteroatoms, e.g., oxygen, sulfur, selenium, phosphorus, and the like.

In many preferred embodiments, the control target nucleic acids are structurally as similar as possible to the test target nucleic acids that are employed in the assay, e.g., both sets of target and control nucleic acids are labeled cDNAs or cDNA fragments or synthetic oligonucleotides. In other words, the structure of the control target nucleic acids should be similar to that of the test target nucleic acids in order to maximally imitate the hybridization of the test target nucleic acids with which they are to be employed.

Each target nucleic acid of the control set should bind to its corresponding probe nucleic acid with selectivity and sensitivity. A nucleic acid that selectively binds with its corresponding probe nucleic acid is at least 10 times, preferably at least 100 times, and more preferably at least 1000 times more likely to bind with its designated probe nucleic acid than to a non-specific nucleic acid, and preferably any other sequence present on the array. Non-specific nucleic acids include those of random sequence, coding sequences found in a particular array other than the designated probe nucleic acid, and coding sequences of non-probe sequences specific to the organism from which the probe nucleic acids are derived.

Control target nucleic acids of the invention also display sufficient sensitivity upon binding with their designated probe nucleic acids. By “sufficient sensitivity” is meant that binding of the probe nucleic acid is significantly greater than the binding of background nucleic acids of random sequence, where the strength of binding is at least 10 times, preferably at least 100 times, and more preferably 500 times greater than the recognition of non-specific or background nucleic acids of random sequence. In many preferred embodiments, the nucleotide sequences of the subject control target nucleic acids are chosen with algorithms, where such algorithms are described in detail in PCT publication WO 97/10365 and PCT/US96/14839, the disclosures of which are herein incorporated by reference.

A feature of the control sets of target nucleic acids is that they include at least one target nucleic acid complementary to each probe nucleic acid present in at least a subset of the probe nucleic acids present on the array with which they are used. In other words, at least a subset of the probe nucleic acids present on a given array are represented in the control set intended for use with the given array, where the probe members of the subset preferably correspond to genes for which quantitative analysis is desired. By at least a subset is meant that at least 20, usually at least 30 and more usually at least 50 of the probe nucleic acids present on the array are represented in the control set. In certain embodiments, at least 20%, usually at least 30% and more usually at least 50% of the probe nucleic acids present on the array are represented in the array. In many preferred embodiments, all of the probe nucleic acids present on the array are represented in the control set. In these preferred embodiments, there is a different nucleic acid member present in the control set which hybridizes under stringent conditions, as defined infra, to each probe on the array. For example, where a given array includes 500 distinct probe nucleic acids which are distinct from each other based on sequence, a control set for use with this particular array includes at least 500 different target nucleic acids of different sequence—one for each probe nucleic acid on the array.

Non-probe sequences on the array may not have a target nucleic acid in the control set, e.g., array sequences such as orientation sequences, negative and positive control sequences, etc. that may be present on an array. In general, control target nucleic acids are not necessary for sequences on an array which do not require quantification, where a particular protocol is intended to provide qualification data only.

The number of unique control target nucleic acids (where any two sequences are; unique if they differ from each other in terms of sequence, where the difference may be a minimal as a 1 base difference) in the set or pool of control target nucleic acids will, in most embodiments, be at least about 8, 10, 20, 50, 100, 200 or more where the number may be as high as about 1,000; 20,000 or higher, but in many embodiments will not exceed about 10,000; 5,000, 800, or 750.

Of particular interest in many embodiments of the invention are control sets that include a representative or representational number of target nucleic acids. As the subject control sets comprise a representational number of target nucleic acids, the total number of different target nucleic acids in any given set will be only a fraction of the total number of different or distinct RNAs in the sample from which the test set of target nucleic acids (described infra) is derived, where the total number of target nucleic acids in the control-set will generally not exceed 80%, usually will not exceed 60-50% and more usually will not 40-20% of the total number of distinct RNAs in the original sample from which the test set of target nucleic acids is derived, e.g. the total number of distinct messenger RNAs (mRNAs) in the original sample. Any two given RNAs in a sample will be considered distinct or different if they comprise a stretch of at least 100 nucleotides in length in which the sequence similarity is less then 98%, 90%, 85%, 80%, 75%, 70%, 65%, 60%, 55%, 50% or 45% or lowere, as determined using the FASTA program (default settings). As the sets of control target nucleic acids comprise only a representational number of target nucleic acids compared to the mRNA population of the sample from which the test set is derived, with sources comprising from 5,000 to 50,000 distinct RNAs, the number of different target nucleic acids in the control set typically ranges from about 20 to 40,000 or 20 to 10,000, usually from about 50 to 2,000 or 50 to 30,000 and more usually from about 100 to 20,000 and sometimes from about 75 to 1500. In the preferred embodiment the representative number of target nucleic acid can be generated using a mixture of gene-specific primers, as described in 08/859,998; 08/974,298; 09/225,998; the disclosures of which are herein incorporated by reference.

Control target nucleic acids can be the same length, shorter or longer than their corresponding probe sequences on the array or test nucleic acid in the solution (if present). However, each control target nucleic acid should have a least partial complementarity to its corresponding probe nucleic acid and at least partial sequence identity with its corresponding test target nucleic acids (if present). By partial sequence identity is generally meant at least about 75%, usually at least about 80% and more usually at least about 90%, and up to and including 100% sequence identity. Preferably, there is no cross-hybridization between any two given control target nucleic acids, such that any two-given control target nucleic acids in a set will not substantially hybridize to the same probe or the complement sequence of the same target under stringent conditions, as defined infra. In addition, the control target nucleic should have structural and hybridization characteristics very similar to its corresponding test target nucleic acid, i.e., it should have similar hybridization efficiencies, similar kinetics with complementary probe sequences, similar non-specific background hybridization with other sequences, etc. For example, where the control set of target nucleic acids comprises labeled cDNAs reverse transcribed from a control set of a representative pool of synthetic RNAs, the test target nucleic acids will also generally be labeled cDNAs reverse transcribed from mRNAs, e.g., synthetic mRNAs. In another embodiment, the test set of nucleic acids can be synthetic oligonucleotides derived from mRNA isolated from a biological sources. For example, the test set may be a mixture of antisense oligonucleotides (ranging in length from about 40 to 120 nt) which could hybridize with mRNA to produce a duplex mixture which is then separated from singlestranded nucleic acids, e.g., by chromatography, precipitation, centrifugation, enzymatic digestion, etc. As a result, the remaining duplex or bound oligonucleotide probe fraction corresponds to the mRNA composition and can be employed as the test probe set. In this case, the control set is preferably a mixture of similar size and sequence antisense oligonucleotides mixed in known concentrations. The compositions of the test and control sets use for hybridization with mRNA will be substantially the same and complementary to probe sequences on the array.

In certain embodiments, substantially similar hybridization efficiencies are ensured by employing control target nucleic acids selected using the protocols described in U.S. patent application Ser. No. 09/440,829; the disclosure of which is herein incorporated by reference. In this protocol, each oligonucleotide of the control set should be chosen so that is capable of hybridizing to a region of the corresponding probe nucleic acid. Different methods may be employed to choose the specific region of the probe to which the oligonucleotide probe is to hybridize. Thus, one can use a random approach. However, instead of using a random approach, a rational design approach may also be employed to choose the optimal sequence for the control sequence in view of the hybridization array with which it is to be employed. Preferably, the sequence of the oligonucleotide probe is chosen based on the following criteria. First, the sequence that is chosen as the probe specific sequence should yield control target that does not cross-hybridize with, or is homologous to, any other oligonucleotide probe for other spots present on the array that do not correspond to the same target. Second, the sequence should be chosen such that the oligonucleotide target has a low homology to a nucleotide sequence found in any other gene, whether or not the gene is to be represented on the array from the same species of origin. As such, sequences that are avoided include those found in: highly expressed gene products, structural RNAs, repeated sequences found in the RNA sample to be tested with the array and sequences found in vectors. A further consideration is to select sequences which provide for minimal or no secondary structure, structure which allows for optimal hybridization but low non-specific binding, equal or similar thermal stabilities, and optimal hybridization characteristics. A final consideration is to select sequences that give rise to targets which efficiently hybridize to their corresponding probes and do not suffer from substantial non-specific hybridization events. Finally, all of the target sequences of the control collection are preferably chosen such that they exhibit substantially the same hybridization efficiency to their corresponding probes, where the difference in hybridization efficiency between any two targets and their corresponding probes preferably does not exceed about 10 fold, more preferably does not exceed about 5 fold and most preferably does not exceed about 3 fold.

Targets meeting the above criteria can be designed or identified using any convenient protocol. A representative protocol includes the following algorithm which is part of the present invention. In selecting targets according to this representative algorithm or process, a unique gene-specific or target specific sequence (one or more regions per gene) is first identified based on a sequence homology search algorithm described in detail in copending application Ser. No. 09/053,375, the disclosure of which is herein incorporated by reference. In this step, the sequence of all genes represented on the to be produced array and all sequences deposited in GenBank are searched in order to select mRNA fragments which are unique for each mRNA or target to be represented on the array. A unique sequence is defined as a sequence which at least does not have significant homology to any other sequence on the array. For example, where one is interested in identifying suitable 80 base long unique probes, sequences which do not have homology of more than about 80% to any consecutive 40 base segment of any of the other probes on the array are selected. This step typically results in a reduced population of candidate target sequences as compared to the initial population of target sequences identified for each specific probe/gene/mRNA of interest.

Of this reduced population of candidate sequences, screening criteria are employed to exclude non-optimal sequences, where sequences that are excluded or screened out in this step include: (a) those with strong secondary structure or self-complementarity (for example long hairpins); (b) those with very high (more than 70%) or very low (less than 40%) GC content; (c) those with long stretches (more than 6) of identical consecutive bases or long stretches of sequences enriched in some motifs, purine or pyrimidine stretches or particular bases, like GAGAGAGAY, GAAGAGAA; and the like. This step results in a further reduction in the population of candidate target sequences.

In the next step, sequences are selected that have similar melting temperatures or thermodynamic stability which will provide similar performance in hybridization assays with target nucleic acids. Of interest is the identification of targets that can participate in duplexes whose melting temperature exceeds 65, usually at least about 75 and more usually at least about 80EC.

The final step in this representative design process is to select from the remaining sequences those sequences which provide for low levels of non-specific hybridization and similar high efficiency hybridization with complementary target molecules. This final selection is accomplished by practicing the following steps:

1. The remaining set of targets which is identified for each probe using the above steps, where this remaining set typically includes at least 1 potential target, usually at least 2 potential targets and more usually at least 3 potential targets, are experimentally characterized for their hybridization efficiency and propensity to participate in non-specific hybridization events using the following protocol.

2. First, an array of at least a portion of the candidate targets (which, since they are attached to the substrate are pseudo “probes” for each probe to be represented on the final array is produced. For example, where three candidate targets have been identified for a particular probe sequence, these targets are attached to the surface of a solid support, along with candidate targets for other probes, to produce a test target array.

3. Next, a normalization control probe set is prepared, wherein each probe in the set is complementary to one target sequence in the array and the various probe constituents of the set are mixed in similar or identical amounts. The number of probes in the set of control probes is usually less than the set of targets in the array. Usually the number of probes in the control set is between 50% and 90%, but can be between 10 and 100%, of the number of test targets (i.e. pseudoprobes) on the array surface. As such, not all of the target sequences on the test array will have a corresponding or complementary probe in the probe control set. For example, where three different candidate targets have been identified for each of 10 different mRNA probes, a test target array of 30 different oligonucleotide targets is prepared. Next, a control set of probe nucleic acids which includes probes that correspond to 5 of the 10 different mRNA targets represented on the array is produced, where the control set includes a probe that is complementary to each different target corresponding to 1 of the 5 different mRNAs represented in the control probe set, i.e. the control probe set includes 15 different probes—1 probe for each of the 15 targets on the array that correspond to the 5 different mRNAs represented in the control probe set. (While the above procedure has been described in terms of using a probe population that corresponds to less than all of the targets on the array so that non-specific hybridization can be determined, other protocols also may be employed. For example, one may use a population of probes that corresponds to all of the targets on the array, where at least a portion of the probes are distinguishable from the remaining portion or portions, e.g. by label, mass etc. Following hybridization, the probes hybridized to each target can be detected and both the efficiency of the target for its true probe and its propensity for non-specific hybridization can be determined).

4. Following generation of the control set of probes, the control set is hybridized with the test target array under stringent conditions and hybridization signals are detected. The intensity of the signal for those targets which have a corresponding labeled complementary probe in the hybridization solution is used as a measure for determining the hybridization efficiency of that target, as well as differences in hybridization efficiency of different candidate targets for different probes. For those targets on the array which do not have complementary labeled probe sequences in control set, the intensity of hybridization signal generated by each of these targets is used to identify the level of non-specific hybridization that characterizes these targets.

5. The above steps are repeated with one or more additional control sets of probe nucleic acids in order to get comprehensive information concerning the hybridization efficiency and level of non-specific hybridization for each candidate of the candidate targets on the array. The number of different sets of control probes that are employed in this process is generally at least two, more commonly at least four and most commonly at least ten.

6. From the above steps, target sequences meeting the following criteria are identified for use as targets in the control sets of the subject invention. First, candidate targets that exhibit a high efficiency of hybridization for their corresponding probes are identified. In many embodiments, candidate targets having substantially the same hybridization efficiency for their respective probes are identified, where any two targets to different probes have substantially the same hybridization efficiency for their respective probes if the differences in hybridization efficiency of the two targets does not exceed 10-fold, where differences of less than about 5-fold and often less than about 3-fold are preferred. Of these identified targets, targets that show substantial cross hybridization or non-specific hybridization are excluded, where a probe that shows non-specific hybridization of up to at least 5-fold, more commonly 20-fold and most commonly 50-fold less than the level of gene-specific hybridization between the target and its corresponding probe are excluded in this step. In other words, in the above assay hybridizations, those targets that exhibit a signal that is at within 5-fold less, usually at least 20-fold less and more usually within 50-fold less of the signal generated by targets and their complementary probes are excluded as being targets with unacceptably high propensities for participating in non-specific hybridization events.

The above algorithm or process is used to design the control targets of the control sets of the subject invention. Steps 1 to 6 can be repeated if, in the first round of selection for particular probes no candidate targets were identified.

Each target nucleic acid in the control set may be the same length as its corresponding probe nucleic acid, longer than its corresponding probe nucleic acid or shorter than its corresponding probe nucleic acid. In general, the length of each target nucleic acid in a given control set is at least about 25 nucleotides, usually at least about 50-60 nucleotides, and sometimes at least about 100 nucleotides, where the length could be as a long as 2 kb or longer, but will generally not exceed about 1 kb and more usually will not exceed about 800 nucleotides.

In many embodiments, a feature of control sets of target nucleic acids is that the concentration of each control target nucleic acid present in the set be known. In other words, the amount of each individual control target nucleic acid in the control set is known. For example, an equal weight amount of each distinct control target nucleic acid may present in the mixture. In other embodiments, an equal molar amount or equimolar amount of each control target nucleic acid may be present. In yet other embodiments, different known amounts or ratios of the various constituent control target nucleic acids may be present. However, in any set of control target nucleic acids employed according to the subject invention, the amount of each constituent member present in the control set is known, either in absolute terms or in terms relative to each other.

The control sets of target nucleic acids are further characterized in that at least two different gene functional classes are represented in a given control set, where the number of different functional classes of genes represented in the a given control set will generally be at least 3, and will usually be at least 5. In other words, the sets of control target nucleic acids comprise nucleotide sequences complementary to RNA transcripts of at least 2 gene functional classes, usually at least 3 gene functional classes, and more usually at least 5 gene functional classes. Gene functional classes of interest include oncogenes; genes encoding tumor suppressors; genes encoding cell cycle regulators; stress response genes; genes encoding ion channel proteins; genes encoding transport proteins; genes encoding intracellular signal transduction modulator and effector factors; apoptosis related genes; DNA synthesis/recombination/repair genes; genes encoding transcription factors; genes encoding DNA-binding proteins; genes encoding receptors, including receptors for growth factors, chemokines, interleukins, interferons, hormones, neurotransmitters, cell surface antigens, cell adhesion molecules etc.; genes encoding cell-cell communication proteins, such as growth factors, cytokines, chemokines, interleukins, interferons, hormones etc.; and the like.

Of particular interest are control sets of target nucleic acids in which each of the genes collectively listed in the tables of the following applications are represented in the control set: U.S. patent application Ser. No. 08/859,998; U.S. patent application Ser. No. 08/974,298; U.S. patent application Ser. No. 09/225,998; U.S. application Ser. No. 09/221,480; U.S. application Ser. No. 09/222,432; U.S. application Ser. No. 09/222,436; U.S. application Ser. No. 09/222,437; U.S. application Ser. No. 09/222,251; U.S. application Ser. No. 09/221,481; U.S. application Ser. No. 09/222,256; U.S. application Ser. No. 09/222,248; and U.S. application Ser. No. 09/222,253; the disclosures of which are incorporated herein by reference.

Another critical feature of the control target nucleic acids is that they are synthetic nucleic acids and not isolated from a biological source. The control target nucleic acids may be generated using any convenient protocol, including reverse transcription protocols (e.g. using AMV or MoMLV reverse transcriptase), bacteriophage RNA polymerase (T7 RNA polymerase, T3, RNA polymerase, etc.) mediated transcription, PCR protocols, oligonucleotide synthesis protocols (i.e. nucleotide chemistry), and the like. In a preferred embodiment, the control target nucleic acid sequences are generated using cDNA fragments cloned into appropriate expression vectors using a set of a representative number of gene specific primers, as described U.S. application Ser. Nos.: 08/859,998; 08/974,298; 09/225,998; the disclosures of which are incorporated herein by reference. These cloned cDNAs are then used to produce RNA control targets using techniques such as PCR and/or bacteriophage RNA polymerase mediated transcription. Of particular interest are applications in which the gene specific primers used to generate the control sets are the same as the gene specific primers used to generate the probe nucleic acids on the array with the control set is employed.

In another preferred embodiment, the control target set is a mixture of syntheitc oligonucleotides where each constituent ranges in size from about 20 to 120 nt, in many cases from about 50 to 100 nt or 60 to 90 nt, and are complementary to mRNAs or cDNAs and to probe sequences on the array.

After synthesis, each control target nucleic acid is quantitated using procedures such as spectrophotometry, fluorescence measurement, etc. Known quantitative amounts of each control target nucleic acid are then mixed together to produce the subject control sets of target nucleic acids, e.g., for use in the hybridization assays, described in greater detail infra. In a preferred embodiment, the control target nucleic acids are mixed together in equal molar amounts, at predetermined ratios, at equal weight or molar amounts, etc, where in many embodiments they will be mixed together in equal weight amounts, such that the amount of each individual target nucleic acid in the control set is the same as any every other individual target nucleic acid in the set.

Test Target Nucleic Acids and Sets Thereof

Turning now to the test target nucleic acids, in many embodiments the test target nucleic acids are generally isolated from a biological sample (cells, tissues, organs, etc.) preparation, and then converted to other nucleic acids using known in the art technology, such as PCR, reverse transcription, hybridization, etc., e.g. mRNA, cDNA, PCR products, cRNA, oligonucleotides, and the like. The target nucleic acids may be isolated from a tissue or cell of interest using any method known in the art. Total RNA or its transcriptionally active fraction mRNA can be isolated from a tissue and labeled and used directly as a target nucleic acid, or it may be converted to a labeled cDNA, cRNA, etc. via methods such as reverse transcription, transcription and/or PCR. Generally, such methods will employ the use of oligonucleotide primers, and the primers can be anchored by bacteriophage RNA polymerase promoter. The primers may be designed to copy a large spectrum of RNA species, e.g. oligo(dT) primers or random hexamers, or designed specifically to copy a subset of genes of interest. After the copying step, i.e. conversion of mRNA to cDNA, cDNA can be amplified by PCR or by linear amplification using bacteriophage RNA polymerase mediated transcription. As with the control target nucleic acids, in a preferred embodiment the test target nucleic acid sequences are generated using a set of a representative number of gene specific primers, as described U.S. application Ser. Nos.: 08/859,998; 08/974,298; 09/225,998; the disclosures of which are incorporated herein by reference.

In an alternative embodiment, the set of test target nucleic acids is produced using a control set of target nucleic acids as follows. In this alternative embodiment, an initial nucleic acid population from a sample of interest, generally an initial mRNA population from a sample of interest is contacted with a control set of target nucleic acids (as described above), where the control set of target nucleic acids is made up of a plurality of distinct nucleic acids of known sequence, where each distinct nucleic acids is present in a known amount. The particular nucleic acids present in the control set are those that correspond to the genes to be assayed, e.g., those that hybridize under stringent conditions to mRNAs of the same genes that are present on the array being used in a given assay. For example, in a protocol where the expression of 500 different genes is to be assayed using an array displaying 500 different probes (one corresponding for to each probe on the array), one for each gene to be assayed, the control set that is contacted with the mRNA from the cell to be assayed includes 500 different control target nucleic acids for which the sequence and amount of each constituent nucleic acid member is known, e.g., where all of the different control target nucleic acids are present in equimolar amounts in the control set.

Contact occurs under hybridization conditions, preferably stringent hybridization conditions such as those described infra, producing a hybridization mix. Contact occurs such that duplex structures are produced between any complementary (typically perfectly complementary) mRNA/control target nucleic acid pairs present in the hybridization mix. Suitable hybridization conditions under which contact may be performed are described infra.

Contact under stringent hybridization conditions, as described above results, in the production of a population of single stranded nucleic acids and duplex structures of mRNAs hybridized to their complementary control target nucleic acids present in the initial control set of target nucleic acids. These duplex structures are then separated from the single stranded nucleic acids present in the hybridization mixture, which components will include non-hybridized mRNAs present in the original sample, non-hybridized control target nucleic acids present in the original control set, etc. Separation may be by any convenient means, including separation based on physical criteria, e.g., size separation such as by electrophoresis, chromatography, e.g., using oligo dT beads which bind complex polyA+ RNA with hybridized control targets (as exemplified in the Experimental Section, infra), centrifugation, selective precipiation, etc. Alternatively, chemical separation means, e.g., chemical crosslinking of single stranded or double stranded fraction, enzymatic separation means, etc., may be employed. For example, an enzyme or enzyme mix that degrades single stranded nucleic acids but not double stranded nucleic acids, e.g., one or more single stranded nucleases, may be employed, where representative enzymes of interest include, but are not limited to: ribonuclease A, —Tl, —B, —I, mung beand nuclease, S1 nuclease; and the like.

The above separation step results in a set of duplex structures. The duplex structures are made up of mRNAs from the initial sample which are hybridized to control target nucleic acids present in the initial control set of target nucleic acids. The expression profile of the initial sample is preserved in this resultant set of duplex structures, in that the amount of each of the mRNAs present in the initial sample is preserved in the set of duplex structures, assuming an excess amount of control target nucleic acid for each mRNA of interest is present in the initial control set. For example, in an initial sample of mRNAs where the mRNA of gene 1 is present in a copy number of 1,000, the copy number of the mRNA of gene 2 is 2,000 and the copy number of the mRNA of gene 3 is 3,000, the copy of the duplex under conditions of saturation hybridization of mRNA with control set of target nucleic acids with similar hybridization efficiencies that includes the mRNA of gene 1 will be 1,000, the copy number of the duplex that includes the mRNA of gene 2 will be 2,000 and the copy number of the duplex that includes the mRNA of gene 3 will be 3,000; assuming that in the initial control set, each of the nucleic acids is present in an excess amount, e.g., at least 1,000 for gene 1, at least 2,000 for gene 2 and at least 3,000 for gene 3, where the amount of excess is generally at least about 5 number %, usually at least about 50 number % and often at least about 100 number % or higher.

The hybridized control target nucleic acids are then separated from their corresponding mRNAs using any convenient protocol. For example, the above resultant set of duplex structures may be treated to separate the duplex structures, e.g., by heating to dissociate the annealed strands, to produce a population of single stranded nucleic acids made up of the mRNAs and the control target nucleic acids. These two components may then be separated from each other using any convenient protocol. For example, since the control target nucleic acids are generally shorter than their corresponding mRNAs, size separation protocols may be employed. Alternatively, where the control target nucleic acids are different nucleic acids from the mRNAs, e.g., where the control target nucleic acids are DNAs, enzymes that selectively degrade RNAs may be employed to achieve separation.

Separation as described above results in the production of set of nucleic acids which may be used as the test set of target nucleic acids in hybridization protocols, just as the test set of target nucleic acids described above may be employed. In the resultant set, because of the method employed to produce it, the quantity of original mRNAs and therefore the expression level of the genes of interest is preserved in the quantities of each individual control target nucleic acid present in the resultant test set. For example, as described above with respect to the example concerning genes 1, 2 and 3, the copy number for the mRNAs for each of these genes in the original sample is the same as or proportional to the copy number for each of the target nucleic acids corresponding to these genes in the test set. As such, if the control set of nucleic acids has similar hybridization efficiencies under hybridization conditions used, the resultant test set provides a true expression profile of the initial sample with respect to mRNAs represented in the control set that is initially employed. Put another way, the resultant set of test target nucleic acids substantially mirrors the initial mRNA sample in terms of quantities of each distinct mRNA present in the sample.

The above described protocol has been discussed in terms of generation of test target from an initial mRNA sample for clarity purposes only. In alternative embodiments, derivatives of the initial mRNA sample like cDNA, etc., may be employed, as described above and as is well within the abilities of those of skill in the art.

Using the above described alternative protocol to prepare the test set of target nucleic acids provides a number of distinct advantages. First, the test set of target nucleic acids is produced without the use of PCR or primer extension reactions. Second, the test target nucleic acids will have substantially the same, if not the same, hybridization efficiency as compared to the control set of target nucleic acids with which they are employed.

Additional Features of the Control and Test Sets of Target Nucleic Acids

Depending on the particular assay protocol with which the subject test and control sets of target nucleic acids are employed, the test and control sets of target nucleic acids may be labeled with the same label, such that the test and control sets cannot be distinguished from one another, or the test and control sets of target nucleic acids may be differentially labeled, such that the two sets are readily distinguishable from each other.

As such, in certain embodiments, the test and control sets of target nucleic acids are differentially labeled. By “differentially labeled” is meant that the test and control sets of target nucleic acids are labeled differently from each other such that they can be simultaneously distinguished from each other. For example, where one has a control set of target nucleic acids and a test set of target nucleic acids, each target nucleic acid in the test set will be labeled with the same first label and each target nucleic acid in the control set will be labeled with the same second label that is different and distinguishable from the first label. Likewise, where two control sets are employed in the method, each target nucleic acid in the second control set will be labeled with a third label different and distinguishable from both the first and second label.

In yet other embodiments, the test and control sets of target nucleic acids are labeled with the same label, so as to be indistinguishable from each other. When the test and control sets of target nucleic acids are labeled with the same label, each target nucleic acid of each set is labeled with the same label.

A variety of different protocols may be used to generate the labeled target nucleic acids, as is known in the art, where such methods typically rely on the enzymatic generation of labeled target nucleic acid using an initial primer and template nucleic acid. Labeled primers can be employed to generate the labeled target. Alternatively, label can be incorporated into the target nucleic acid during first strand synthesis or subsequent synthesis, labeling or amplification steps in order to produce labeled target. Label can also be incorporated directly to mRNA using chemical modification of RNA with reactive label derivatives or enzymatic modification using labeled substrates. Representative methods of producing labeled target are disclosed in U.S. application Ser. Nos.: 08/859,998; 08/974,298; 09/225,998; the disclosures of which are incorporated herein by reference.

For synthetic oligonucleotides target label(s) can be incorporated during oligonucleotide synthesis, post synthesis chemical or enzymatic labeling.

A variety of different labels may be employed, where such labels include fluorescent labels, isotopic labels, enzymatic labels, particulate labels, etc. For example, suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), cyanine dyes, e.g. Cy5, Cy3, BODIPY dyes, e.g. BODIPY 630/650, Alexa542, etc. Suitable isotopic labels include radioactive labels, e.g. ³²P, ³³P, ³⁵S, 3H. other suitable labels include size particles that possess light scattering, fluorescent properties or contain entrapped multiple fluorophores. The label may be a two stage system, where the target DNA is conjugated to biotin, haptens, etc. having a high affinity binding partner, e.g. avidin, specific antibodies, etc. The binding partner is conjugated to a detectable label, e.g. an enzymatic label capable of converting a substrate to a chromogenic product, a fluorescent label, and isotopic label, etc.

Any combination of labels, e.g., first and second labels, first, second and third labels, etc., may be employed for the test and control target sets, provided the labels are distinguishable from one another. Examples of distinguishable labels are well known in the art and include: two or more different emission wavelength fluorescent dyes, like Cy3 and Cy5, or Alexa 542 and Bodipy 630/650; two or more isotopes with different energy of emission, like ³²P and ³³P; labels which generate signals under different treatment conditions, like temperature, pH, treatment by additional chemical agents, etc.; and labels which generate signals at different time points after treatment. Using one or more enzymes for signal generation allows for the use of an even greater variety of distinguishable labels based on different substrate specificity of enzymes (e.g. alkaline phosphatase/peroxidase).

Array Hybridization

The next step in the subject methods is the hybridization of the test and control sets of target nucleic acids to a nucleic acid array(s). The nucleic acid arrays employed in the subject hybridization assays have a plurality of nucleic acid spots, and preferably in many embodiments oligonucleotide or polynucleotide spots, stably associated with a surface of a solid support, where the solid support may be rigid, e.g. glass, or flexible, e.g. nylon membrane or plastic film. At least a portion of the nucleic acid spots on the array are made up of probe nucleic acids. Arrays with which the subject methods find use include: nucleic acid biochips, e.g. cDNA biochips, RNA biochips, polynucleotide biochips, oligonucleotide biochips, and the like. Of particular interest are the arrays described in: U.S. patent application Ser. No. 08/859,998; U.S. patent application Ser. No. 08/974,298; U.S. patent application Ser. No. 08/859,998; U.S. patent application Ser. No. 08/974,298; U.S. patent application Ser. No. 09/225,998; U.S. application Ser. No. 09/221,480; U.S. application Ser. No. 09/222,432; U.S. application Ser. No. 09/222,436; U.S. application Ser. No. 09/222,437; U.S. application Ser. No. 09/222,251; U.S. application Ser. No. 09/221,481; U.S. application Ser. No. 09/222,256; U.S. application Ser. No. 09/222,248; U.S. application Ser. No. 09/222,253; U.S. Application Serial No. 60/104,179; the disclosures of which are incorporated herein by reference.

As mentioned above, in practicing the subject methods the test and control sets of target nucleic acids are hybridized to an array, where the sets of target nucleic acids may be hybridized to the same array or different arrays, where when the sets of target nucleic acids are hybridized to different arrays, all of the different arrays will at least share common arrays of probe nucleic acids, i.e., they will be identical with respect to their probe nucleic acids.

In certain preferred embodiments, the test and control sets of target nucleic acids are hybridized to the same array. In such embodiments, the array is hybridized with a test set of labeled target nucleic acids and at least one control set of labeled target nucleic acids. In those embodiments where more than one control set of target nucleic acids is employed, the number of different control sets may range from 2 to 6, usually 2 to 4 and more usually 2 to 3. Of particular interest are those embodiments in which 1 or 2 different control sets of target nucleic acids are employed.

The probe and control sets of target nucleic acids may be hybridized to the array and/or detected simultaneously or sequentially. Thus, where a control set and target set are employed, the two sets of target-nucleic acids may be combined prior to hybridization and the array hybridized to both simultaneously to minimize potential variability in hybridization conditions. For example, a known amount of labeled sets of test target and control target nucleic acids can be added to the same hybridization buffer, and then contacted with one or more arrays simultaneously under hybridization conditions. In another example, a known amount of labeled sets of test target and control target nucleic acids are added to the same hybridization mix, and this buffer aliquoted for the separate hybridization of different arrays. By storing aliquots of the hybridization mix (e.g. storage at −20EC or −70EC), different arrays may be hybridized at different times with approximately the same amounts of target nucleic acid sequences.

In the above embodiments where the test and control target nucleic acids are hybridized simultaneously to a given array, labeled test and control target nucleic acids are premixed or pooled prior to contact with the array. In a preferred embodiment, mixtures of test and control target nucleic acids have amounts of control and target nucleic acids which are sufficient to generate signals that are at least 10 fold, usually at least 20 fold and more usually at least 50 fold higher than background signals observed with the array. The relative amounts of control and test target nucleic acids in the mixture are selected to be sufficient to allow reliable detection of the test sequences complimentary to the probe nucleic acid while at the same time allowing complete binding of the test target nucleic acids with a nofold excess of unbound probe nucleic acid on the array. The amount of test nucleic acid present in the mixture is usually determined by available amount of RNA sample and sensitivity of technology employed in a particular protocol. Preferably, the amount of test nucleic acid present in the mixture ranges from about 0.01-100 (g of nucleic acid, e.g. cDNA, and more usually from about 0.1-10 (Dg of nucleic acid, e.g. cDNA. In many embodiments, the amount of control target nucleic acid employed in the hybridization protocol is about the same or less than the amount of test target nucleic acid that is employed, where less than typically means 10 fold less, usually 100 fold less and more usually 1000 fold less. Of interest are mixtures of labeled nucleic acids that provide for an intensity of signal from each probe nucleic acid in the control detection channel that ranges from about 0.001-0.1%, usually from about 0.001 to 0.01% abundance level.

Alternatively, one or more arrays may be hybridized with the control and test sets of target nucleic acids sequentially. For example, arrays may be hybridized with a hybridization mix containing the labeled test target nucleic acids to allow these molecules uninhibited access to the probe sequences of the array. Following this hybridization, control target nucleic acids could then be exposed to the array for use as an internal control. The hybridization of the control target nucleic acids may be completely separate from the hybridization of the test target nucleic acids, e.g. using different hybridization mixes at different times, or the control target sequences may be added to the hybridization buffer containing the test target nucleic acids following an incubation period with the test target nucleic acids. The latter is especially appropriate when the test target nucleic acids require a longer hybridization incubation period than the control test nucleic acids. When used sequentially, the control and test target nucleic acids may be differentially labeled or labeled with the same label, since detection occurs separately.

In another embodiment, the control set of target nucleic acids is hybridized with several arrays in a “lot” of prepared arrays for quality control purposes. Following quality control characterization, the rest of the arrays for the same lot can be used for hybridization with the a test set and data generated at the quality control step used to normalize data generated with the test set, thereby providing for great efficiencies in the use of the array.

In yet other embodiments, the test and control sets of target nucleic acids are hybridized to different arrays, where each of the different arrays has an identical population of probe sequences, i.e. the different arrays do not vary with respect to their probe sequences. In such methods, the control and test target nucleic acids may be labeled with the same label so as to be indistinguishable from one another, and discussed above. In certain embodiments, the test and control target nucleic acids are hybridized to high throughput array devices, as described in 5,545,531 and PCT/US99/00248, the disclosures of which are herein incorporate by reference.

The control and test sets of target nucleic acids are hybridized to the array(s) by contacting the control and test sets with the array(s) under hybridization conditions. By “hybridization conditions” is meant conditions sufficient to promote Watson-Crick hydrogen bonding to occur between the target and probe nucleic acids. The hybridization conditions, such as hybridization time, temperature, wash buffers used, etc. can be altered to optimize the efficient and specific binding of the target sequences. Test target nucleic acids having sequence similarity to the probes may be detected by hybridization under low stringency conditions, for example, at 50° C. and 6×SSC (0.9 M sodium chloride/0.09 M sodium citrate, 1% SDS) and remain bound when subjected to washing at 55° C. in 1×SSC (0.15 M sodium chloride/0.015 M sodium citrate, 1% SDS). Test target sequences with sequence identity may be determined by hybridization under stringent conditions, for example, at 60° C. or higher and 6×SSC (0.9 M sodium chloride/0.09 M sodium citrate, 1% SDS). Preferably, the control target nucleic acids have a region of substantial identity to the provided probe sequences on the array, and bind selectively to their respective probe sequences under stringent hybridization conditions. Other suitable hybridization conditions for various nucleic acid pairs are well known to those skilled in the art and reviewed in Maniatis et al., and in PCT WO 95/21944.

Analysis of the differences in signal generated by two or more sources may be carried out by using multiple arrays with the same or similar probe compositions, one for each set of test target nucleic acids. Each array is then hybridized with a labeled set of control target nucleic acids and a labeled set of test target nucleic acids. Preferably, the labeling efficiency and amount of control target sequences and test target sequences is approximately equivalent between arrays, e.g. an equal amount of labeled control target nucleic acids is used to hybridize to each array. This is not essential, however, since hybridization of the set of labeled control target nucleic acids functions as an independent internal control for each probed array.

Levels of hybridization of test target RNA to the probe compositions can be standardized by comparing the hybridization signal of the test with control target sequences on each array. Differences in hybridization of the control target sequences allows a comparison of relative hybridization levels between arrays.

Washing

Following hybridization, non-hybridized labeled nucleic acid is removed from the support surface, conveniently by washing, generating a pattern of hybridized nucleic acid on the substrate surface. A variety of wash solutions and protocols are known to those of skill in the art and may be used. See Sambrook, Fritsch & Maniatis, Molecular Cloning: A Laboratory Manual (Cold Spring Harbor Press)(1989).

Detection

The resultant hybridization patterns of labeled nucleic acids may be visualized or detected in a variety of ways, with the particular manner of detection being chosen based on the particular label of the target nucleic acid, where representative detection means include scintillation counting, autoradiography, fluorescence measurement, colorimetric measurement, light emission measurement, light scattering and the like.

Following detection or visualization, the hybridization patterns generated by control and test target nucleic acids may be compared to identify differences between the signals. Where arrays in which each of the different probes corresponds to a known gene are employed, differences in signal intensity can be related to a different target concentration of a particular gene (or more specifically copy number of mRNA for a particular gene, i.e., expression level of a particular gene). The comparison of the intensity of binding of a test target nucleic acid to a probe sequence can be compared to the intensity of the binding of the corresponding control target sequence, and the measurement converted to a quantitative RNA concentration for that target sample. The quantitative RNA levels of the test target can be compared between arrays to identify or confirm differential expression of genes in particular samples.

UTILITY

The subject methods find use in, among other applications, standardization of differential gene expression assays. Thus, one may use the subject methods in the differential expression analysis of: (a) diseased and normal tissue, e.g. neoplastic and normal tissue, (b) different tissue or tissue types; (c) developmental stage; (d) response to external or internal stimulus; (e) response to treatment; and the like. The methods of the subject invention therefore find use in broad scale expression screening for drug discovery, diagnostic and research applications, such as the effect of a particular active agent on the expression pattern of genes in a particular cell, where such information can be used to reveal drug toxicity, carcinogenicity, etc., environmental monitoring, disease research and the like. A number of different tasks can be accomplished with the subject invention, which tasks include, but are not limited to: detecting relative hybridization of target sequences, calibrating a hybridization assay, harmonizing data between hybridization assays, ensuring quality control of arrays; and testing reagents used in a hybridization assay. The subject methods in which control and test sets of target nucleic acids are employed can also be used in the generation of gene expression databases, as the data generated from the subject methods are quantitative, reflect the real RNA concentration rather than intensity of signal, and are independent of the type of array. Each of these different aspects of the invention is discussed separately below.

Detecting Relative Hybridization of Target Sequences

The methods of the present invention are useful in detecting relative levels of hybridization of different genes in a sample by providing a set of internal hybridization controls. Since the control set of nucleic acids are of a known sequence, in a known quantity, and of a known specific activity (where in a preferred embodiment the control and test target are labeled with the same specific activity), the level of hybridization of the control nucleic acids can be used to determine the level of expression of each gene in a test sample based on its level of binding to a probe sequence. The fact that each probe sequence has its own internal control also allows for the detection of potential expression differences between samples and differences in binding affinities between probe sequences, both on a single array and between arrays. Thus, the intensity level of hybridization of a control sequence can be used to calculate the expression level of a gene in a sample based upon the intensity of the test target hybridization to the corresponding probe sequence. A feature of this method is that the calculate expression level is a relative rather than absolute number (intensity) and therefore more reproducible from experiment to experiment, etc.

Calibrating a Hybridization Assay

The methods of the subject invention also find use in the calibration of hybridization assays. Using known concentrations of probe nucleic acid, test target nucleic acids, and control target nucleic acids allows one to optimize the hybridization conditions for a particular use, such as increasing stringency to allow better detection of nucleic acids with some level of sequence homology (e.g. differential expression between genes from a single family or alternative splice forms for the same gene). The use of the internal standards of the method of the subject invention allows hybridization, labeling procedures, and the like to be optimized for a particular use, which is especially valuable for standardization of large scale of hybridization assays, such as high-throughput screening of biological samples. Optimization thus means that one can change hybridization conditions in order to achieve maximal intensity of specific hybridization signals with complimentary probe sequences and minimal level of non-specific hybridization with non-complementary probe sequences.

Harmonizing Data Between Hybridization Assays

The methods of the subject invention also find use in the harmonization of data between hybridization assays, thus allowing for a direct comparison of expression levels despite potential differences due to variables such as differences in hybridization conditions, differences in sample preparation and even between different types of arrays, differences in quality and performance within and between different arrays, differences in specific activity of the labeled target sequences, differences in array quality, and the like. Because each hybridization assay has its internal control for at least a subset of the probe sequences on the array, the data can be compared using ratios of the intensity of the control target nucleic acids and the intensity of the test target nucleic acids. Thus, the use of simple mathematical formulations to correct for differences between assays allows the levels of gene expression in these different assays to be adjusted to the same level and then compared in a biologically relevant fashion.

Testing Reagents and Equipment, Optimizing Hybridization/Wash Conditions, Used in a Hybridization Assay

The methods of the present invention are also useful in determining the efficacy of hybridization reagents. Such reagents may be, for example, new reagents, e.g. different buffer solutions for prehybridization and hybridization, or established reagents, e.g. a new batch of a known, commercially available reagent. The internal control of the methods of the subject invention provide for two levels of quality assurance upon testing the reagents, basically providing an extra control for determining the efficacy of a reagent in a single hybridization. Efficiency means maximum specific signal with minimal level of non-specific signal and background binding to solid surface. Other parameters such as temperature, buffer composition, length of hybridization and/washing times, etc., may be optimized using calibration controls. Also, the same calibration target nucleic acids can be used routinely to test and calibrate detection equipment to expected level intensity of signals, thus limiting variability due to functionality of the equipment; and may be used to test and calibrate the quality of arrays for control procedures. Specifically, the control sets of target nucleic acids can be employed to ensure the quality of arrays for use in hybridization assays, by ensuring that each probe spot on the array actually contains the intended probe sequence and the quality of these sequences at the intended spatial location. For example, to ensure the quality of an array of 100 different probe sequences corresponding to 100 different genes, a control set in which each of the 100 different genes is represented may be contacted with the array. Any spots to which target from the control set does not hybridize are then identified as defective, whereby the quality of the array is tested. As such, the subject invention also provides arrays that have been processed by this quality assurance protocol, such that the arrays are known to have all of the intended probe sequences displayed on their surface. In other words, arrays are provided by the subject invention in which the sequence of each probe on the array is known and is known to hybridize to a complementary target in solution. The data generated in the quality assay protocol are specific for each lot of arrays and can be used to adjust expression data generated using test set of targets for different lots or types of arrays.

Building Gene Expression Databases

The subject methods in which control and test target nucleic acids are employed can be used to determine real RNA concentrations in sample. This information can in turn be used to compile a gene expression database. Use of subject methods in building a database has advantages over databases derived from images or signal intensities. Such advantages include: the generation of more compact information (number/versus image file); the identification of expression levels that are not dependent on type of array, hybridization conditions, lot of array, etc. These advantages are significant in that expression data obtained with the subject methods does not need-annotation-to-be meaningful; and the database generated from the data can be universal, i.e. it can be generated using data generated in different labs, or at different times, or even using different types of arrays.

Also provided are databases of gene expression profiles produced as described above. The subject expression profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. One format for an output means ranks toxicity profiles possessing varying degrees of similarity to a reference toxicity profile. Such presentation provides a skilled artisan with a ranking of similarities and identifies the degree of similarity contained in the test toxicity profile.

The subject expression profile databases find use in a number of different applications. For example, where one has an expression profile of interest, one can search the database to determine whether that profile is present in the database and, if so, readily identify the source of the expression profile, i.e., the identify of the sample that has the given expression profile.

The comparison of an expression profile obtained from an assayed sample and expression profiles present in the database, i.e. reference expression profiles, is accomplished by any suitable deduction protocol, AI system, statistical comparison, etc. Methods of searching databases are known in the art. See, for example, U.S. Pat. No. 5,060,143, which discloses a highly efficient string search algorithm and circuit, utilizing candidate data parallel, target data serial comparisons with an early mismatch detection mechanism. For other examples, see U.S. Pat. No. 5,720,009 and U.S. Pat. No. 5,752,019, the disclosures of which are herein incorporated by reference.

KITS

The present invention also provides kits for performing the subject array-based hybridization assays. The subject kits at least include a control set of target nucleic acids, as defined above, or a precursor thereof. By “nucleic acid precursor” is meant any nucleic acid from which with the control set may be prepared, e.g., a set of RNAs encoding the nucleic acids of the control set, plasmids containing nucleic acids for generation of the control set, and the like. Labeled cDNA can be derived from these precursors by enzymatic synthesis, or oligonucleotides chemically synthesized based on sequence information of these precursors. The set may contain RNAs that recognizes each probe composition on an array, and such RNAs may be pre-labeled, may be labeled for use with the test target nucleic acids, or may be converted to labeled cDNA for hybridization. Kits of the present invention may also contain cDNA or oligonucleotides that selectively binds to the probe compositions of the array to be screened. The cDNAs or oligonucleotides may be pre-labeled, or may be labeled by the user through any convenient protocol, such as the protocol used to generate the labeled test target nucleic acids. A kit containing a set of control target RNAs may further contain oligonucleotides for the production of cDNA. In a preferred embodiment, these oligonucleotides are gene specific primers, particularly gene specific primers that have sequence identical to those that were used in the production of the probe compositions on the array to be used in the particular assay. In another embodiment, primers can be oligo dT or random primer, if these primers are used for making test sample target.

The subject invention also provides arrays precalibrated in a quality control laboratory protocol using a control set of target nucleic acids, as described above. These arrays together with data of control set hybridization corresponding to the array are employed by the user to noramlize hybridization efficiency of the test set during actual use of the arrays.

Kits for carrying out differential gene expression analysis assays are preferred. Such kits according to the subject invention will at least comprise the subject sets of nucleic acids. The kits may further comprise one or more arrays corresponding to the set of control target nucleic acids. In many such embodiments, each gene represented on the array is also represented in the control set of target nucleic acids. Of particular interest are the arrays disclosed in: U.S. patent application Ser. No. 08/859,998; U.S. patent application Ser. No. 08/974,298; U.S. patent application Ser. No. 08/859,998; U.S. patent application Ser. No. 08/974,298; U.S. patent application Ser. No. 09/225,998; U.S. application Ser. No. 09/221,480; U.S. application Ser. No. 09/222,432; U.S. application Ser. No. 09/222,436; U.S. application Ser. No. 09/222,437; U.S. application Ser. No. 09/222,251; U.S. application Ser. No. 09/221,481; U.S. application Ser. No. 09/222,256; U.S. application Ser. No. 09/222,248; U.S. application Ser. No. 09/222,253; U.S. Application Serial No. 60/104,179; the disclosures of which are incorporated herein by reference.

The kits may further comprise one or more additional reagents employed in the various methods, such as: primers for generating target nucleic acids; dNTPs and/or rNTPs, which may be either premixed or separate; one or more uniquely labeled dNTPs and/or rNTPs, such as biotinylated or Cy3 or Cy5 tagged dNTPs; or other post synthesis labeling reagents, such as chemically active derivatives of fluorescent dyes, enzymes such as reverse transcriptases, DNA polymerases, RNA polymerases and the like; various buffer mediums, e.g. hybridization and washing buffers; prefabricated probe arrays; labeled probe purification reagents and components, like spin columns, etc.; signal generation and detection reagents, e.g. streptavidin-alkaline phosphatase conjugate, chemifluorescent or chemiluminescent substrate; and the like.

In addition to the sets of nucleic acids, arrays and other components described above in the general description of kits, the assay kit may further include a set of gene specific primers that are employed to generate labeled targets. In many embodiments, the set of gene specific primers will be the same primers used to generate the polynucleotide probes that are present on the array to be screened. Of particular interest in certain embodiments are kits comprising a set of primers selected from the primers identified as SEQ ID NO: 01-1372, where in these kits of particular interest, at least twenty, usually at least 50 and more usually at least 100 of the gene specific primers in the kit will be selected from this group of primers identified as SEQ ID NO: 01-1372, as shown in Table 1 of U.S. patent application Ser. No. 08/859,998, the disclosure of which is herein incorporated by reference.

The following examples are offered by way of illustration and not by way of limitation.

### EXPERIMENTAL

### EXAMPLE 1

### Identification of Differentially Expressed Genes

An assay to determine relative levels of gene expression in a sample is conducted as follows.

A. Preparation of Human Stress cDNA Array

236 cDNA fragments corresponding to 236 different human genes were amplified from quick-clone cDNA (CLONTECH) in 236 separate test tubes using a combination of sense and antisense human stress gene-specific primers as described in U.S. patent application Ser. No. 09/222,256, the disclosure of which is herein incorporated by reference. Amplification was conducted in a 100-μl volume containing 2 μl of mixture of 10 Quick-clone cDNAs from placenta, brain, liver, lung, leukocytes, spleen, skeletal muscle, testis, kidney and ovary (CLONTECH), 40 mM Tricine-KOH (pH 9.2 at 22EC), 3.5 mM Mg(OAc)₂, 10 mM KOAc, 75 μg/ml BSA, 200 μM of each dATP, dGTP, dCTP and dTTP, 0.2 μM of each sense and antisense gene-specific primers and 2 μl of KlenTaq Polymerase mix. Temperature parameters of the PCR reactions were as follows: 1 min at 95EC followed by 20-35 cycles of 95EC for 15 sec and 68EC for 2 min; followed by a 10-min final extension at 68EC. PCR products were examined on 1.2% agarose/EtBr gels in 1×TBE buffer. As a DNA size marker a 1 Kb DNA Ladder was used. Double stranded (ds) cDNA was then precipitated by addition of a half volume of 4M ammonium acetate (about 35 μl) and 3.7 volumes of 95% ethanol (about 260 μl). After vortexing, the tube was immediately centrifuged at 14,000 r.p.m. in a microcentrifuge for 20 min.

The pellet was washed with 80% ethanol without vortexing, centrifuged as above for 10 min, air dried, and dissolved in 10 μl of deionized water. Yield of ds cDNA after the amplification step was about 5 μg. The ds cDNA fragments for all 236 genes were cloned into pBR322 derived vectors using convention blunt end cloning procedure and identity of the clones was confirmed by sequence analysis. The ds cDNA inserts with the sequence corresponding 236 genes were amplified by PCR using a combination of antisense and sense gene-specific primers, as described above. The ds cDNA was denatured by boiling in alkaline buffer (pH 8-10)(sodium bicarbonate 300 μM), or DNA was dissolved in 5 M guanidinium isothionate. All cDNA probes were transferred in a 384-well plate and deposited onto a glass slide using process analogous to that described in PCT Application Serial No. PCT/US99/00248, the disclosure of which is herein incorporated by reference.

B. Isolation and Preparation of Test Target Ribonucleic Acids from Human Tissue Sample

As described in greater detail below, the above described human array is used to screen a human tissue sample to determine gene expression of each of the 236 genes in a human sample. The tissue sample is tested for expression by isolating mRNA from the sample and subsequently transcribing the mRNA into labeled cDNA. This labeled pool of cDNA is then used as test target nucleic acids for hybridization against the probe nucleic acids on the array.

Total RNA is isolated from homogenized human tissue using a ATLASPURE□ RNA isolation kit and-mRNA is isolated from the total RNA using an oligo-(dT)-cellulose spin column (both from CLONTECH, Palo Alto Calif.) according to the manufacturer's protocols. The mRNA of the tissue sample is used to generate target cDNAs for hybridization to the probe array.

C. Preparation of Control Target Ribonucleic Acids

A control set of ribonucleic acids was synthesized by T7 transcription from cDNA fragments corresponding to the 236 genes on the Human Stress array prepared as described above, where each of the cDNA fragments included a. T7 promoter. The fragments were amplified by PCR in 236 separate tubes as described in section A using a combination of sense and antisense gene specific primers. Antisense primers carry a T7 promoter for following transcription resulting in RNA fragments mimicking the corresponding mRNA fragment. T7 transcription was accomplished in 236 separate tubes in a 200 Φl volume containing 200 ng of the individual cDNA fragment, 80 mM HEPES-KOH (pH 7.5 at 22EC), 15 MM MgCl₂, 2 mM spermidine, 10 mM DTT, 3 mM each ATP, GTP, CTP, UTP, and 200 units of T7 RNA polymerase (Epicentre Tech., Madison, Wis.). Incubation of reaction mixture was performed at 37 EC for 2 hours. RNA products where examined on a 2% agarose/EtBr gel in 1 HTAE buffer. RNA was then precipitated by addition of equal volume of 4M ammonium acetate and 2 volumes of 95% ethanol. After vortexing, the tubes were immediately centrifuged at 14,000 rpm for 20 min. The RNA pellet was dissolved in 100 Φl of water and purified by column chromatography using a CHROMA SPIN-200 column (CLONTECH, Palo Alto, Calif.) according to the manufacturer's instructions.

After purification, the concentration of each RNA fragment sample was determined on a spectrophotometer by UV absorption with 1 optical density unit corresponding to 40 Φg of RNA. The adjusted equal molar weight concentration for each fragment was then calculated. A length of 2200 nucleotides (which is equal to the average length of mRNA) was used as a standard. For example, if the real concentration for a particular fragment with a size of 200 nucleotides was 1 ng/μl, its adjusted equal molar weight concentration was 11 ng/μl. Then all 236-synthetic RNAs were mixed in equal molar ratio and this mixture of synthetic RNAs was used as a standard, as described below.

D. cDNA Synthesis

2 μl of RNA dissolved in water at 0.5-1 μg/μl (or appropriate concentration for the control set of RNA) was mixed to 1 μl of 1 OHCDS primer mix (0.2 mM each, comprising mixture of antisense primers complementary to 236 mRNAs related to the Stress/toxicology array). The sample in the tube was then mixed by vortexing and spun down by microcentrifuge. The tubes were then incubated in a prewarmed PCR thermal cycler at 70EC for 2 min. The temperature was then reduced to 50EC and incubation was continued for another 2 min. The remaining reagents were then added and incubation was continued for 20 min. The final reaction conditions were: 50 mM Tris-HCl pH 8.3, 75 mM KCl, 3 mM MgCl₂, 0.5 mM each of dGTP, dCTP, dATP, 0.1 mM of dTTP and 0.1 mM of allylamine-dUTP, 5 mM DTT, 50 units of MMLV Reverse Transcriptase. Reaction was stopped by the addition of 1 Φl of termination mix (0.1 M EDTA pH 8.0; 1 mg/ml Glycogen). cDNA was precipitated by the addition of an equal volume of 4M ammonium acetate and 2 volumes of ethanol. The pellet was collected by centrifugation, washed with 70% ethanol, dried and dissolved in water.

E. Labeling of Control Target and Test Target Nucleic Acids

The control target nucleic acids and the test target nucleic acids are labeled using differentially detectable fluorescent labels. For labeling of the test target nucleic acids, following conversion of the target ribonucleic acids to amino-modified cDNAs, 1 mg of Cy3 succinimide ester is dissolved in 10 Φl of dimethyl sulfoxide, and 10 Φl of the test target cDNAs are added to it. For labeling of the control target set, 1 mg of Cy5 succinimide ester is dissolved in 10 Φl of dimethyl sulfoxide and 10 Φl of control target cDNAs are added to it. Both mixtures are incubated at room temperature overnight.

Each labeled set of target nucleic acids is purified separately by column chromatography using a CHROMA SPIN-200 column (CLONTECH, Palo Alto, Calif.) according to the manufacturer's instructions. See also U.S. patent application Ser. No. 08/859,998, the disclosure of which is herein incorporated by reference.

F. Contact of the Target Sequences to the Array

The control target and test target cDNAs are then pooled for hybridization to the array. To prepare the mixture of labeled control and test target cDNAs, 1 μg of the Cy3 labeled test target cDNA and 1 μg of the Cy5 labeled control target prepared above are combined.

G. Hybridization and Detection of Probe Sequences on Array

A solution of ExpressHyb (CLONTECH) and sheared salmon testes DNA (Sigma) is prepared as follows: 5 ml of ExpressHyb□ is prewarmed at 50-60EC. 0.5 mg of the sheared salmon testes DNA is heated at 95-100EC for 5 min, and then chilled quickly on ice. Heat-denatured sheared salmon testes DNA is mixed with prewarmed ExpressHyb□. The human cDNA array is placed in a hybridization container, and 1 ml of the ExpressHyb□/salmon DNA solution is added. Prehybridization is conducted for 5 min with continuous agitation at 60EC. Labeled cDNA test and control target nucleic acids, as prepared above, (about 200 Φl) are mixed with 2 Φl (1 Φg/Φl) of human Cot-I DNA, and denaturated at 99EC for 2 min. The mixture is added to the hybridization solution and mixed together thoroughly. The container is sealed by sealing tape. Hybridization is allowed to proceed overnight with continuous agitation at 60EC. The hybridization solution is carefully removed and discarded in an appropriate container, and replaced with 10 ml of Wash Solution 1 (2H SSC, 0.1% SDS). The array is washed for 10 min with continuous agitation at 65EC. The step is repeated two times. Additional 10-min washes are performed in 10 ml of Wash Solution 2 (0.1H SSC, 0.1% SDS) with continuous agitation at 65EC. Using forceps, the cDNA array is removed from the container, briefly washed in 0.1 HSSC and excess buffer is removed from surface by centrifugation in a Beckman CS-6R centrifuge at 2000 rpm. Glass arrays are scanned using a confocal scanning microscope (General Scanning, Watertown, Mass.). Images are scanned at a resolution of 10 μm per pixel.

### EXAMPLE 2

### Determination of Differential Expression Between Samples

A method to determine differentially expressed genes in normal and cancerous cells, or treated versus untreated cells, is conducted as follows.

Two identical human cDNA arrays and a set of control target cDNAs are prepared using the methods as described in Example 1.

Two separate sets of test target nucleic acids are produced, one set isolated from a cancerous human tissue, and the second set isolated from the corresponding physiologically normal human tissue. Each tissue sample is tested for differential expression by isolating mRNA from each sample and subsequently transcribing the mRNA into labeled cDNA. Each labeled pool of cDNA is then used as test target nucleic acids for hybridization against the probe sequences of one of the two arrays, and the levels of expression between the two arrays is compared to determine relative differences in expression between the normal and the cancerous tissues.

Following preparation, both the set of control target nucleic acids and the two sets of test target nucleic acids, corresponding to the normal and the cancerous tissue, are labeled using detectable fluorescent labels. Each set of test target nucleic acids is labeled in a separate reaction with Cy3. For each labeling procedure, an aliquot of test cDNAs is added to a 0.5 ml Eppendorf tubes containing 1 mg of Cy3 succinimide ester dissolved in 10 Φl of dimethyl sulfoxide. For differential labeling of the control target set, 1 mg of Cy5 succinimide ester is dissolved in 10 Φl of dimethyl sulfoxide and 10 Φl of cDNA generated from the control set PCR reaction is added to it. All of these mixtures are incubated at room temperature overnight. Each labeled set of target nucleic acids is purified separately by column chromatography.

Each array is hybridized using one of the two labeled sets of test target nucleic acids, and one-half of the labeled set of control target nucleic acids. A solution of ExpressHyb□ (CLONTECH) and sheared salmon testes DNA (Sigma) is prepared and prewarmed at 55EC. 1 mg of the sheared salmon testes DNA is heated at 95-100 EC for 5 min, and then chilled quickly on ice. Heat-denatured sheared salmon testes DNA is mixed with the prewarmed ExpressHyb™, and 1 ml of the ExpressHyb™/salmon DNA solution is added to each of two separate hybridization containers. Prehybridization is conducted for 5 min with continuous agitation at 60EC.

Each hybridization mixture is prepared by mixing about 200 Φl of one set of test target cDNAs synthesized from 1 Φl of polyA RNA, 100 Φl of the set of control target cDNAs synthesized from 1 Φg of control RNA mix containing 0.01% of each synthetic control RNA for each probe on the array (the size of each synthetic RNA is adjusted to average size of mRNA, i.e., 2.2 kb mixed together and then equal molar weight % was adjusted to 0.01%), and 2 Φl (1 Φg/Φl) of human Cot-I DNA. The mixture is denatured at 99EC for 2 min prior to hybridization. The hybridization mixture containing the test nucleic acids from the cancerous tissue is added to the prehybridization solution of the first array, and the hybridization mixture containing the test nucleic acids from the normal tissue is added to the prehybridization solution of the second array. Each array is also contacted with the labeled control set of target nucleic acids. Each container is sealed, and hybridization conditions and detection are carried out as described in Example 1.

Direct comparisons of the amounts of expression on each array are carried out by determining the ratio of the intensity of the test target hybridization with the intensity of the control target hybridization. Once determined, these ratios allow for correction of experimental variation between the two arrays, and thus allow a direct comparison of the levels of gene expression in the cancerous and non-cancerous samples. A set of housekeeping control probe signals is used in order to adjust the intensity of both channels to each other.

### EXAMPLE 3

Synthetic RNA corresponding to the Human Stress array was synthesized for 236 fragments arrayed on membrane or glass. RNA fragments were mixed in equal molar ratio and this mixture was used for further experiments.

cDNA from a control mixture of RNA was labeled with Cy5 fluorescent dye as described earlier. cDNA from placenta poly(A)+ mRNA or liver poly(A)+ mRNA was labeled with Cy3 dye.

Hybridization was performed on the glass slides. The first probe contained labeled control cDNA. Hybridization of this probe to a glass Human Stress array was done at different concentrations relative to amount of poly(A)+ RNA. Hybridization was to 5 different glass slides at concentrations of 0.01%, 0.04%, 0.1%, 0.4%, and 1%, corresponding to 0.1 ng, 0.4 ng, 1 ng, 4 ng, 10 ng of adjusted weight amount of individual RNA species in the mixture. At the same time, 0.1 ng of each individual control Cy5 labeled cDNA (corresponds to 1%) was mixed with the Cy3 labeled cDNA from 1 Φg poly(A)+ RNA. Hybridization of this sample was performed on a 6^{th} glass slide array. Hybridization conditions were as described earlier. The first five glass slides were used for plotting the calibration curve and the 6^{th} glass slide was use for determination of concentration of RNA in the sample based on calibration curve and intensity of C y3 and Cy5 dyes in the same spot. The corresponding ratio of intensity of Cy5 to Cy3 was determined for all other spots.

In the range of synthetic RNA concentrations used (from 0.01% to 1%), the intensity of the signal in the spot increased proportionally (and linearly) with an increasing amount of RNA. The linear change in intensity of the signal from synthetic RNA in each spot allowed the direct comparison of the abundance of an mRNA of interest on two arrays with liver and placenta poly(A)+ RNA samples using the intensity of the Cy5 channel as a normalization value.

The type of calibration curve is not dependent upon the method of synthesizing the sample or the complexity of the probe (control RNA mix may be used alone or added to poly(A)+ RNA before labeling. This was demonstrated using a membrane based array with radioactive labeling instead of fluorescent dyes. Design of this experiment was similar to the glass experiment described above. Five different concentrations of control RNA were used (from 0.01% to 1%). Labeling of these probes was done separately and after the mixing to 1 Φg poly(A)+ RNA.

### EXAMPLE 4

### Preparation and Use of Test Set of Target Nucleic Acids Generated from Control Set of Nucleic Acids.

The protocol described below illustrates the preparation of a test target set of nucleic acids using a hybridization control set of labeled oligonucleotides with RNA isolated from biological source. The protocol also illustrates the hybridization of these labeled test targets in combination with a differentially labeled control targetset to an oligonucleotide based array to determine relative levels of gene expression in a sample.

A. Preparation of Oligonucleotide Glass Microarray.

A-1. Preparation of Aminopropyl-Glass.

1. Prepare wash solution: to get 2 liters, dissolve 200 g NaOH in 600 ml water and make up volume to 1 liter (20% w/v). To this solution add 1 liter ethanol. This makes 10% NaOH in 50% EtOH. Wash glass in this solution on orbital shaker overnight. (slides are placed in rack)

2. Transfer rack(s) with slides into bath with MilliQ water and wash on shaker for 15-20 min, repeat this step one more time.

3. Transfer slides into bath with acetone and wash on shaker for 15-20 min. Repeat this step two more times. Dispose acetone from first wash and keep acetone from 2nd and 3rd washes. (When doing this procedure again, use 2nd wash as first, 3rd as second and for the 3rd wash use fresh acetone.

4. Prepare in advance 5% solution of water in acetone (5% water B 95% acetone).

5. During last wash step prepare 0.5% solution of aminopropyltriethoxysilane (Sigma, cat No A3648) in acetone-water mixture from step 4.

6. Transfer slides from last acetone wash into silanization solution and incubate for 2 hours at room temperature on orbital shaker.

7. Transfer slides into MilliQ water and wash for 20 minutes.

8. Transfer slides into acetone and wash for 20 min, repeat this step 2 more times. These acetone washes are to be disposed.

9. Preheat oven at 110° C.

10. Remove rack with slides from the last acetone wash and transfer it into preheated oven. As some acetone still remains on slides and on racks surfaces, the smelt becomes quite intensive. Exhaust duct should be open after putting slides into oven and may be closed after-first 30 minutes of baking.

11. Program oven to bake slides at 110° C. for 3 hours and then shut down or cool down to room temperature. It is convenient to do this step overnight.

12. After baking is oven, slides are ready for printing using “thiocyanate method”. If the printing will not be done right away, slides may be kept in clean boxes inside dry cabinets.

The following steps are for preparation of PDITC-slides.

1. Prepare a mixture of Pyridine and Dimethylformamide (10% pyridine and 90% DMF). Prepare only as much as necessary. This mixture cannot be stored.

2. Dissolve 1,4-Phenylenediisothiocyanate in the Pyridine-DMF mixture at 0.1% concentration (1 g per liter) on stirrer. Prepare this solution only as much as necessary and only when ready to proceed with next steps. This solution cannot be stored. The solution should be light yellow-green in color.

3. Pour the solution in a tray and transfer tray(s) with amino-modified slides into the solution. Close the tray with the lid and shake on orbital shaker at low speed for 2 hours.

4. Transfer rack(s) with slides into a tray with acetone and wash on shaker for 10-15 minutes. Repeat this step 2 more times by transferring rack(s) into trays with fresh acetone.

5. After last wash quickly transfer racks with slides into vacuum oven and dry in vacuum at room temperature for 20-30 minutes. Vacuum should be applied as fast as possible.

6. Dispose Pyridine-DMF mixture and acetone washes into flammable wastes container.

7. Transfer slides for storage into dry cabinets. Make sure the desiccant in the dry cabinet is good (blue in color).

A-2. Preparation of Oligonucleotide Probes

91 long sense oligonucleotides corresponding to 91 human genes were synthesized using an automated nucleic acid synthesizer and standard phosphoramidite chemistry (Operon, Alameda, Calif.).

A-3. Printing of Oligonucleotides.

Oligonucleotides used in this experiment were dissolved in 0.1 M NaOH at 100 nanogramm per microliter and printed on PDITC modified glass surface. Amount of DNA deposited was about 5 ng per spot. After printing slides were baked at 80° C. for 2 hours and then UV crosslinked (254 nm UV lamp) for 1 min.

B. Preparation of Test Target Oligonucleotide Set

B-1. Preparation and Labeling of Control Set of Oligonucleotides.

Control set of 91 5′-amino modified antisence oligonucleotides that were substantially complementary to the 91 oligonucleotide probes printed on glass slide microarray (example 4-A) were synthesized using an automated nucleic acid synthesizer and standard phosphoramidite chemistry (Operon, Alameda, Calif.). The set of 91 control oligonucleotides was mixed together at final total concentration 9 μM (0.1 μM of each oligonucleotide) and labeled by Cy3 and Cy5 succinimide ester in separate test tubes and purified using protocol described in example 2 above and reagents from Atlas Glass fluorescent labeling kit (CLONTECH, Palo Alto, USA).

B-2. Preparation of Test Set Oligonucleotide Targets from Control Set of Oligonucleotides.

1 μl of Cy3-labeled control set of oligonucleotides was mixed with 5 μg of placenta poly(A)+ RNA in final volume 10 μl of hybridization buffer, containing 50 mM Hepes-KOH, pH 7.8, 500 mM KCl, 1 mM EDTA, 50% formamide, denaturated at 70° C. for 1 min and hybridized at 50° C. for 2 hr in thermocycler. After hybridization the mix was diluted by 90 μl of binding buffer (50 mM Hepes-KOH, pH 7.8, 500 mM KCl, 1 mM EDTA) containing 1 μl of 5 μM oligo d(T)25 containing biotin group at 5′- and at 3′-end (CLONTECH, Palo Alto, Calif.) and incubated at 50° C. for 5 min, then mixed with 15 μl of Streptavidin magnetic beads (Amersham-Pharmacia, Amersham, England) prewashed in binding buffer and incubated additional 30 min at 50° C. in thermal shaker. Using magnetic separator Streptavidin magnetic beads with bind complex of RNA/Cy3-oligonucleotides were washed 3 times by 500 μl of washing buffer (50 mM Hepes-KOH, pH 7.8, 150 mM KCl, 0.1 mM EDTA) at room temperature 20° C. and Cy3-oligonucleotide test set was eluted in 50 μl of water at 60° C.

C. Relative Gene Expression Profiling in Placenta RNA Using Test Set and Control Set of Oligonucleotide Targets.

50 μl of Cy3-labeled oligonucleotide test set generated at step B-2 was mixed with 0.1 μl of Cy-5 labeled oligonucleotide control set generated at step B-1, denatured at 95° C. for 1 min, mixed with 2 ml of GlassHyb hybridization solution (CLONTECH) and hybridized overnight at 55° C. with glass microarray generated at step A-3. Then glass microarray was washed 3 times for 10 min each in 10 ml of 1×SSC, 0.1% SDS, then rinsed in 0.1×SSC, dry and scanned in Axon microarray scanner. The ratio between Cy3 and Cy5 channels was used in order to calculated relative expression level of 91 human genes expressed in human placenta RNA.

It is evident from the above results and discussion that the subject invention provides a significant contribution to the field of array-based gene expression analysis. With the subject invention, quantitation data is obtained from a single array and therefore errors due to variables in array quality, array type, assay conditions, etc., that are seen in situations involving two or more arrays, e.g. a control array and a test array, are avoided. Furthermore, data received from different arrays, even different types of arrays, may be compared. In addition, because a known amount of control target nucleic acids is employed in the subject methods and comparison of two different signals from a single array spot is performed, concentration determinations may be made that do not depend on efficiency of hybridization or efficiency of reverse transcription. Moreover, the same set of control target nucleic acids can be used to prepare a test set of targets based on hybridization of control set with mRNA, thereby ensuring substantially identical hybridization efficiencies between control and test target nucleic acids and the probes of the array.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. An array-based hybridization assay comprising the steps of:
contacting an array(s) with a test set of target nucleic acids and a control set of target nucleic acids, wherein said control pool of target nucleic acids comprises at least 20 distinct target nucleic acids, wherein each of said at least 20 distinct target nucleic acids is of known sequence and is present in known amount; and
detecting hybridization patterns of said test and control sets of target nucleic acids to said array(s).

2. The assay according to claim 1, wherein at least a subset of the probe nucleic acids present on said array are represented in said control set of target nucleic acids.

3. The assay according to claim 2, wherein all of said probe nucleic acids present on said array are represented in said control set of target nucleic acids.

4. The assay according to claim 1, wherein said test set of target nucleic acids is labeled with the same label as said control set of target nucleic acids.

5. The assay according to claim 1, wherein said test set of target nucleic acids is labeled with a different label from said control set of target nucleic acids.

6. The assay according to claim 1, wherein said test and control sets of target nucleic acids are contacted with the same array.

7. The assay according to claim 1, wherein said test and control sets of target nucleic acids are contacted with first and second arrays, respectively, wherein each of said first and second arrays display the same probe nucleic acids.

8. The assay according to claim 5, wherein said test and control sets of target nucleic acids are contacted with the same array.

9. The assay according to claim 8, wherein said assay further comprises generating said control set of target nucleic acids.

10. The assay method according to claim 1, wherein said assay further comprises generating said test set of target nucleic acids.

11. The assay method according to claim 10, wherein said test set generation comprises:
contacting an initial nucleic acid sample with a control set of target nucleic acids under conditions sufficient to produce duplex structures made up of complementary sample initial nucleic acids-hybridized to control target nucleic acids of said control set;
isolating said resultant duplex structures; and
recovering control target nucleic acid components of said duplex structures to produce said test set of target nucleic acids.

12. The assay according to claim 1, wherein said control set of target nucleic acids comprises at least 50 distinct target nucleic acids.

13. The method according to claim 1, wherein said method further comprises comparing said test and control hybridization patterns.

14. The assay according to claim 1, wherein said test target nucleic acids and said control target nucleic acids are contacted to said array simultaneously.

15. The assay according to claim 1, wherein said test target nucleic acids and said control target nucleic acids are contacted to said array sequentially.

16. The assay according to claim 1, wherein said assay further comprises calculating individual test target nucleic acid concentrations present in said set of test target nucleic acids.

17. A set of control target nucleic acids for use in hybridization to an array of probe nucleic acids corresponding to a plurality of different genes, wherein said control set comprises at least 20 distinct target nucleic acids, wherein each of said distinct target nucleic acids of said control set is of known sequence and is present in known amount, and at least a subset of said probe nucleic acids on said array is represented in said control set of target nucleic acids.

18. The set of control target nucleic acids according to claim 17, wherein said control target nucleic acids are selected from the group consisting of: ribonucleic acids and deoxyribonucleic acids.

19. The set of control target nucleic acids according to claim 17, wherein each of said probe nucleic acids on said array is represented in said control set of target nucleic acids.

20. A kit for use in an array based hybridization assay, said kit comprising:
(a) an array of probe nucleic acids corresponding to a plurality of different genes; and
(b) a control set of target nucleic acids wherein said control set of target nucleic acids comprises at least 20 distinct target nucleic acids, wherein each of said distinct target nucleic acids of said control set is of known sequence and is present in known amount, and at least a subset of said probe nucleic acids on said array is represented in said control set of target nucleic acids.

21. The kit according to claim 20, wherein said nucleic acids are ribonucleic acids.

22. The kit according to claim 20, wherein said kit further comprises primers.

23. The kit according to claim 22, wherein said primers are gene specific primers.

24. The kit according to claim 20 wherein said kit further comprises a label.

25. The kit according to claim 20, wherein each of said probe nucleic acids on said array is represented in said control set of target nucleic acids.
